(19) United States
(12) Patent Application Publication
(10) Pub. No.: US 2011/0045081 A1
(43) Pub. Date: Feb. 24, 2011

(54) MAGNETIC, PARAMAGNETIC AND/OR SUPERPARAMAGNETIC NANOPARTICLES

(76) Inventors: Benedikt Steitz, Vevey (CH); Alke Fink, Toffen (CH); Jatuporn Salaklang, Lausanne (CH); Andrija Finka, Lausanne (CH); Heinrich Hofmann, Pully (CH)

Correspondence Address: Clifford W. Browning;Krieg DeVault LLP, One Indiana Square, Suite 2800, Indianapolis,, IN, 46204, US

(21) Appl. No.: 12/734,130

(22) PCT Filed: Oct. 12, 2007
(86) PCT No.: PCT/IB2007/054169

§ 371 Date: Nov. 2, 2010

(51) Int. Cl. A61K 9/14; C40B 50/00; C40B 30/00; C07K 1/14; G01N 33/53; C12N 5/071; C12N 13/00; H01F 1/00; B82Y 5/00

(52) U.S. Cl. 424/489; 506/23; 506/7; 530/412; 435/7.1; 435/325; 435/173.1; 252/62.51 R; 977/773; 977/904

(57) ABSTRACT

The present invention relates to nanoparticles having a mean diameter of <500 nm and comprising, at their surface, a selected material. The nanoparticles are taken up by cells under physiological conditions and can be used to isolate interaction partners of the selected material within the cells. The present invention provides important advantages in that it opens up new ways of identifying cellular components and of delivering a substance of interest specifically to a selected cell compartment. The nanoparticles are also useful as a tool of diagnosis and for the constitution of chemical libraries.

### TECHNICAL FIELD

The present invention relates to a method of isolating interacting partners of selected material, a method of obtaining information on characteristics of a selected material, a method of delivering a selected material to within living cells, a method of producing a chemical library, a method of diagnosis, a method of producing nanoparticles and nanoparticles obtained by the method.

### PRIOR ART AND THE PROBLEM AND OBJECTIVES UNDERLYING THE INVENTION

The targeting and identification of interaction partners of a selected molecule within living cells is a major task in biotechnological, pharmaceutical, and diagnostic development. Today, methodology for identifying and isolating interaction partners of cellular components, such as sugars, peptides, DNA, RNA, proteins, and so forth, is laborious. This is especially true when it comes to identifying the interaction partner of a given compound under physiological conditions, that is, within the living cell.

A number of approaches have been used to investigate protein interactions. Many of them such as co-precipitation and co-purification remove the proteins from their normal environment (T. K. Kerppola Nature Review 2006 449). The study of protein interactions in their normal environment by techniques as fluorescence complementation, fluorescence resonance energy transfer (FRET) or fluorescence cross-correlation spectroscopy is described in literature.

In the last decade, magnetic techniques have been developed for the isolation and purification of cellular components, as is discussed with respect to proteins and peptides, in the review article of Safarik and Safarikova, BioMagnetic research and Technology, 2004, 2:7. In this article it is described that magnetic carriers bearing an immobilized affinity or hydrophobic ligand are mixed with a sample containing target compounds. Such samples may be crude cell lysates, whole blood, plasma, ascites fluid, milk, urine, cultivation media and many others. Following an incubation period when the target compounds bind to the magnetic particles the whole magnetic complex is easily and rapidly removed from the sample using an appropriate magnetic separator. After washing out the contaminants, the isolated target compounds can be eluted and used for further work, such as analysis.

This principle has been and currently still is applied, as evidenced by numerous publications. For example, Chang et al, Analytical Biochemistry 325 (2004) 175-184 use anti-body-conjugated superparamagnetic beads for isolating neutrophil plasma membranes from disrupted cells. Lüers et al., Electrophoresis (1998), 19, 1205-1210, disclose magnetic beads to which an antibody against peroxisomal protein was covalently bound in order to isolate said protein from homogenized liver cells.

Fan et al, Journal of Colloid and Interface Science 266 (2003) 215-218, report the isolation of the protein avidin from its incubation solution by the aid of Fe₂O₃ nanoparticles, said nanoparticles coated with biotin.

In the above-cited references, the isolation of cellular components, such as proteins or small molecules, are accomplished in the absence of living cells, for example in crude cell lysates and solutions. Accordingly, it remains unclear if the ligand attached to the magnetic beads would also bind to the same compound under physiological conditions. This is disadvantageous in so far as it is not clear if an interaction between the ligand attached to the magnetic beads and the isolated cellular component would also occur within living, intact cells. In particular, certain binding processes between a cellular component and a ligand of interest can be energy-dependent and, thereby only occur in the intact cell. Furthermore, some binding processes can be confined to specific intracellular compartments.

It is an objective of the present invention to provide a way for identifying binding and interaction partners of a material, such as a ligand of interest. In particular, it is an objective of identifying and/or isolating cellular interaction partners of such a ligand. It is also an objective of the present invention to provide a way for isolating cellular components, such as entire cell organelles or molecules, for example, from cell material.

A further objective of the present invention is to provide a tool for studying and visualizing interactions between a ligand of interest and cellular components under physiological conditions. In this context, it is an objective of the present invention to provide means for studying and identifying structural relations of molecules within cells under physiological conditions.

Another objective of the present invention is the delivery of a selected material to a specific organ and/or tissue of the human or animal body, preferably to specific cells, and more preferably to specific compartments within the cells.

A still further objective of the present invention is to provide a way of incorporating magnetic, paramagnetic or superparamagnetic particles into living cells. More preferably, it is an objective to be able to control the mechanism of uptake of the particles by the cells. For example, in some instances uptake of nanoparticles within vesicles surrounded by a membrane (endocytosis), is not desirable, since targeting within cells may be more difficult. Accordingly, it is an objective of providing particles comprising potential binding partners of cellular components, whereby said particles are taken up by the cell by a specific cell uptake mechanism.

Another objective of the present invention is to provide an efficient tool for constituting chemical libraries, such as protein libraries of cells.

A yet other objective of the present invention is to provide new means of diagnosing the absence or presence of a specific condition, such as the presence or absence of a medical condition such as a disease state, of a condition of stress, of a status within the cell cycle and/or of a developmental status.

It is a further objective of the present invention to provide functionalised nanoparticles, in particular nanoparticles carrying a plurality of different functions at their surface. In this context, it is an objective of the present invention to provide nanoparticles that can be targeted to specific cell compartments and/or cell organelles.

It is also an objective of the present invention to study, visualize and/or identify transport ways and interaction partners of nanoparticles in the cell.

### SUMMARY OF INVENTION

Remarkably, the inventors of the present invention found that cells incubated with nanoparticles having a mean diameter of <500 nm, preferably <300 nm, said nanoparticles comprising a magnetic, paramagnetic or superparamagnetic core and comprising, at their surface a material of interest, such as a ligand of interest, were taken up by intact cell. Surprisingly, the uptake of the particles could be controlled. Furthermore, the inventors could target the nanoparticles to specific cell compartments, the transport of the nanoparticles within the cells could be imaged and interacting partners of the nanoparticles could be isolated.

Accordingly, in a first aspect, the present invention provides a method of isolating one or more interacting partner of one or more selected material, said method comprising the steps of exposing living cells to nanoparticles, wherein said nanoparticles have a mean diameter of <500 nm, comprise a magnetic, paramagnetic or superparamagnetic core, and, comprise, at a surface of said nanoparticle, said selected material, removing said nanoparticles from said cells following exposure to the nanoparticles, and separating the nanoparticles bound to said interacting partner of the selected material by the aid of magnetic and/or electric forces, thereby isolating said interacting partner.

In a second aspect, the present invention provides a method of obtaining information on characteristics of a selected material within living cells, said method comprising the steps of exposing living cells to nanoparticles as defined above, removing said nanoparticles from said cells following exposure to the nanoparticles, separating the nanoparticles by the aid of magnetic forces from said cells, and studying and/or characterising cell components interacting with said selected material, thereby obtaining information on characteristics of the selected material within cells.

In a third aspect, the present invention provides a method of delivering a selected material to within living cells, the method comprising the steps of exposing living cells to nanoparticles, as defined above.

In a fourth aspect, the present invention provides a method of producing a chemical library, the method comprising the steps of isolating binding partners of a selected material according to the invention, and, constituting a library of said binding partners.

In a fifth aspect, the present invention provides a method of diagnosis of a presence and/or an absence of a condition, the method comprising the steps of a) producing a chemical library according to the fourth aspect of the invention, b) comparing the chemical library to a standard library representing a specific condition, and, c) diagnosing from similarity between the chemical library obtained in step a) and the standard library the presence and/or the absence of said condition.

In a sixth aspect, the present invention relates to a method of producing nanoparticles comprising a magnetic, paramagnetic and/or superparamagnetic core and, at a surface of the nanoparticles, at least a first and a second selected material, wherein the first and the second materials are different, the method comprising the steps of preparing a suspension of primary nanoparticles, said primary nanoparticles comprising a magnetic or paramagnetic core; guiding said suspension through a magnetic field; immobilising said primary nanoparticles in said magnetic field; guiding a solution or suspension containing a dissolved or suspended first material over said immobilised primary particles so as to obtain nanoparticles comprising a first material at the surface; guiding a further solution or suspension comprising a dissolved or suspended second selected material over said immobilised primary particles so as to obtain nanoparticles comprising a first and a second selected material.

In a seventh aspect, the present invention relates to nanoparticles as defined herein, for example nanoparticles obtainable by the method of the sixth aspect of the invention.

The present invention also relates to the nanoparticles of the present invention for use as therapeutic and diagnostic tool. For example, the nanoparticles of the present invention may be used as medicaments, for example in drug or substance delivery, gene therapy and/or immunotherapy.

The present invention provides many advantages. In particular:
Magnetic and superparamagnetic nanoparticles can be modified and covered with up to 20 different ligands on the same particle.
Modified nanoparticles can be taken up in the cells depending on the surface functionalization and can be specifically targeted in living cells.
With different combinations of ligands on the nanoparticle surface multifunctional tasks can be performed (fluorescence, radiolabeling). This allows to control uptake mechanism and pathways with the surface grafted ligands.
Living cells can be lyzed while interaction of nanoparticles to binding partners and associated molecules stays intact.
Nanoparticles can be analyzed with respect to their direct binding partners by means of chemical, physical and optical methods, while prior art techniques use cell extract for identification of interaction partners here we use living cell incubation.
Nanoparticles can be analyzed with respect to indirect interaction partners that are close to the interaction site or on cell membranes and organelles by means of chemical, physical and optical methods.
Structural relations of proteins and molecules can be identified.
Energy dependent binding events to nanoparticles can be identified.
Transport pathways of the nanoparticles in the cell, complex forming partners and molecules can be identified.
Creation of small molecules, sugars, peptides, DNA, RNA, protein, antibodies, or chemical libraries on the surface of nanoparticles. These libraries can be screened with respect to their interaction partner in living cells.
Interaction partners of inorganic surfaces and inorganic composite materials can be identified.
Nanoparticle cargo can be delivered intracellularly to the nucleus (DNA delivery) and mitochondria (proapoptotic drugs) can be solved by employing different signaling peptides on a single particle.
Drug and/or genes can be delivered to subcellular compartments with magnetic nanoparticles.

In the drawings,

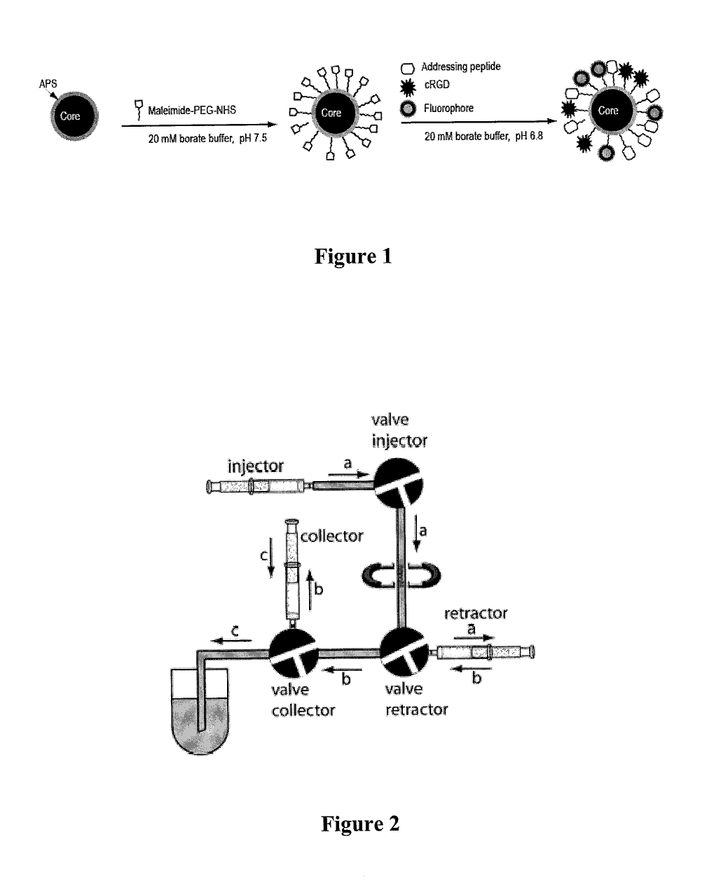

FIG. 1 schematically illustrates the process of preparation and the structure of nanoparticles of the present invention.

FIG. 2 illustrates the functioning of a fixed bed reactor suitable for preparing the nanoparticles. Liquid injected through injector follows the path a) to retractor. Following closing of injector valve, retractor can inject liquid by path b) to collector after which sample is being collected c).

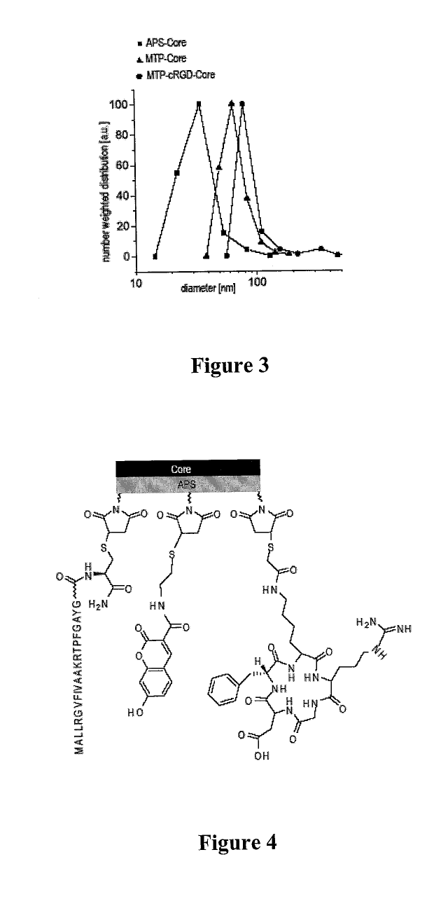

FIG. 3 shows the size distribution of various different nanoparticles produced according to the invention.

FIG. 4 shows nanoparticles functionalised with different selected materials, in particular with a target signal peptide, a fluorescent dye and a cyclic RGD peptide for uptake of the nanoparticle by the cell.

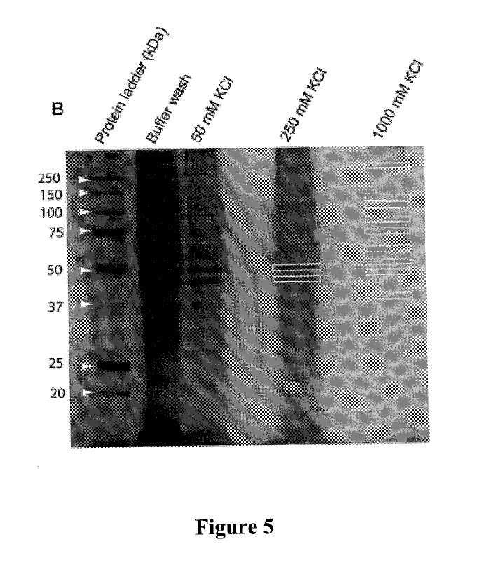

FIG. 5 is a picture of an SDS-PAGE gel with resolved proteins isolated by the method of the present invention, stained by Coomassie-Blue.

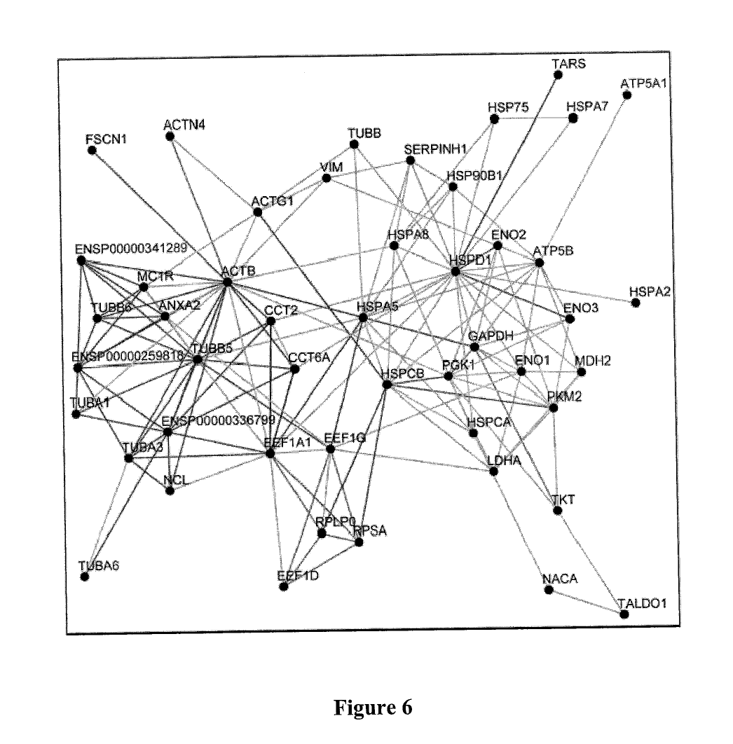

FIG. 6 illustrates physiological and cellular relations of the proteins identified. Analysis was based on STRING algorithm with 0.4 confidence (string.embl.de).

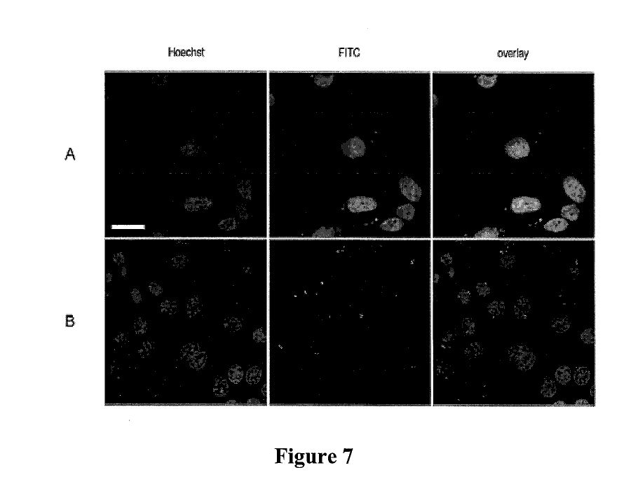

FIG. 7 shows a live cell imaging of cells exposed to nanoparticles targeted to the nuclei of cells. Hoechst stands for a dye added to cells that were incubated with nuclear targeted nanoparticles. “A” shows cells with absorbed particles of Example 4.2.A, whereas “B” shows cells incubated with particles differing from those of Example 4.2.A only in that they are devoid of a cRGD peptide. Both particle types contain FITC fluorescent dye at their surface. Particles without cRGD show reduced uptake and targeting ability.

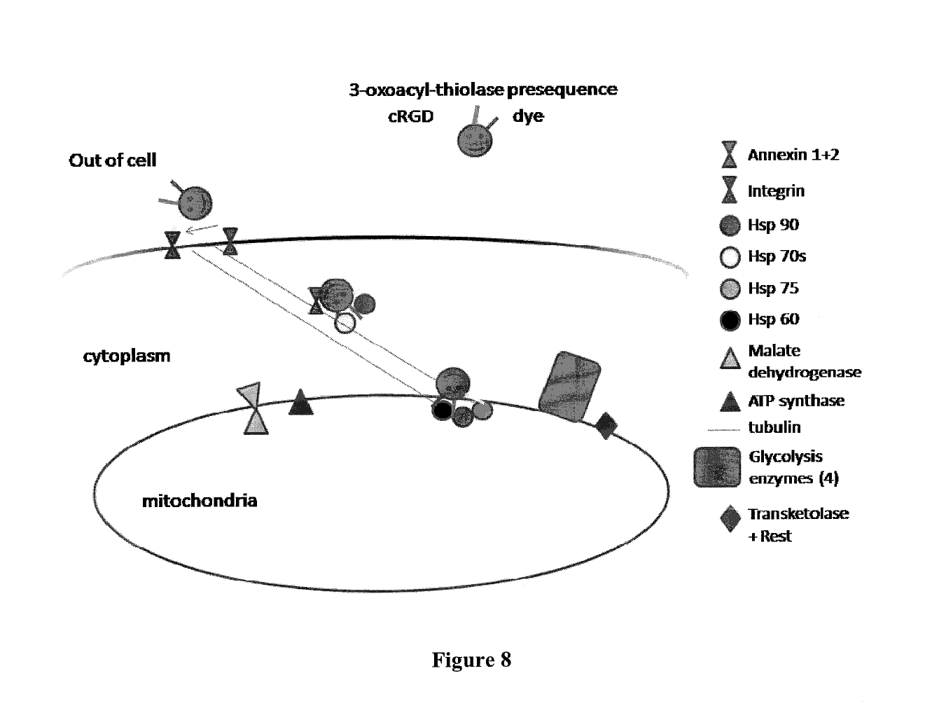

FIG. 8 illustrates schematically the interactions of the nanoparticles and their transport within the cells, as determined from analysis of interacting partners.

### DETAILED DESCRIPTION OF THE PREFERRED EMBODIMENTS

The present invention relates to a method of isolating one or more interacting partner of one or more selected material. An “interaction partner”, in the context of the present invention, preferably is a cellular component, more preferably an intracellular component. For example, the interaction pal tiler may be a molecule, such as a protein, peptide, carbohydrate, DNA, RNA, or a larger cell structure, such as part of or the entire of a cell compartment, for example a cell organelle such as a mitochondrion, a plastid (e.g. chloroplast, chromoplast, leucoplast, and so forth), or larger assemblies of molecules and/or macromolecules that carry out one or several particular functions, but lack membrane boundaries, such as ribosomes, cytoskelleton, and so forth.

A “selected material”, can be any material of interest. For example, the “selected material” can be organic or inorganic. It may be a molecule, but it may also be a quantum dot, for example. An advantage of the present invention resides in that it provides a platform for screening for interaction partners of any substance of interest. For example, an interaction partner may be a specific peptide or protein. Another example of an interaction partner is a bioactive compound, such as a drug. It is clear that these examples, and those provided herein below, are only illustrative of the concept of the invention, which provides a tool to the skilled person for screening for interaction partners of any matter of interest to the skilled person. Accordingly, it is the goal of the present invention that the skilled person defines the exact nature of the “selected material”, generally in dependence of the research that is to be conducted and the information that is whished to be obtained.

An “interacting partner” for the purpose of the present invention, is a component, preferably a cellular component, which in some way interacts with the nanoparticle, in particular with the selected material that is situated at the surface of the nanoparticle. The interaction between the “interacting partner” and the selected material is such that there is at least some sort affinity that creates an association of the selected material and the interacting partner. The association may be mediated by a chemical bond, such as an ionic or covalent bond, or by lower intermolecular forces, such as van der Waal forces, as well as electrostatic interactions, for example.

The methods of the present invention comprise the step of exposing living cells to nanoparticles. The term “comprising”, for the purpose of the present invention, is intended to mean “includes amongst other”. It is not intended to mean “consists only of”.

“Living cells”, for the purpose of the present invention, are metabolically active, intact cells. “Intact cells” are cells that comprise an outer cell membrane boundary. The expression “living cells” does not only include healthy cells, as the present invention also provides a method for diagnosing a condition, such as a disease. More specifically, the expression “living cells” refers to cells that have not been subjected to a man-provoked lysis or desintegration procedure, during which the outer cell membrane barrier has been at least partially destroyed and/or the intracellular cell components have been released from the cell.

In the step of “exposing” living cells to nanoparticles, the term exposing refers to bringing living cells, in particular the outer surface of the cells, in contact with the nanoparticles. According to an embodiment, the step of exposing living cells to nanoparticles comprises a step selected from (i) incubating cells in vitro with said nanoparticles, and (ii) administrating said nanoparticles to a human or animal subject. Accordingly, the term “exposing” refers to both, in vivo and in vitro conditions. In particular, the nanoparticles are exposed to the living cells under physiological conditions.

According to an embodiment, the step of exposing cells to said nanoparticles comprises the step of adding a suspension of nanoparticles to said cells. For example, 1-100 microgram iron/ml suspension or cell medium, respectively, may be added.

Preferably, the nanoparticles are exposed to the cells for a time sufficiently long for allowing the cells getting in contact with the particles and allowing the cells taking the particles up with or without the assistance of an external field. The external field can be electric or magnetic. Depending on the intracellular interaction component to be identified, incubation time may be adjusted so that interaction with the respective component is optimised. In vitro, and also in vivo, incubation time may be in the range of minutes to months. Uptake of the nanoparticles can be verified, for example by the aid of optical methods and/or magnetic methods.

Following incubation, the cells are preferably washed with suitable buffers so as to remove nanoparticles that have not been taken up by the cells.

The present invention comprises the step of removing said nanoparticles from said cells. This step refers to the modification of the cell permeability and/or integrity so as to enable a removal of the nanoparticles and their interacting partners from the cell plasma, the term “cell plasma” referring to all cellular components that are inside the cell plasma membrane, when the cell is intact. In the simplest case, removal of the nanoparticles and their interaction partners from the remaining cell material may be achieved by lysing the cells. Alternatively, the cells may be rendered permeable to the nanoparticles, whereafter the latter may then be removed from the permeable cells.

For example, permeability of the cells can be increased by electroporesis so that particles can be isolated from the cells without destroying the cells. Following the step of rendering the cells permeable, the nanoparticles may be removed from the cells by magnetic forces, for example.

According to an embodiment, the present invention comprises a step of lysing and/or destroying the cells following the step of exposing to the nanoparticles. Lysis may be performed mechanically and/or chemically, for example. In all cases, care should be taken not to destroy the interaction between the nanoparticle surface and cellular interaction components. The destroyed and/or lysed cells obtained in this step may be referred to as “destroyed cells”.

Many techniques are available for cell lysis such as mechanical devices as Waring Blender or Polytron where rotating blades grind and disperse cells and tissue. Liquid homogenization devices where cells or tissue suspensions are sheared by forcing through a narrow space (syringe, Dounce homogenizer, French Press, Potter-Elvehjem Homogenizer). Sonication in a sonicator where high frequency sound waves shear cells. Freeze/Thaw cycles where repeated cycles of freezing and thawing disrupt cells through ice crystal formation. Manual grinding where mortar and pestle are used to homogenize tissue. These methods have been reviewed extensively. Chemical methods include the use of additives and facilitators as cells can be treated with various agents to aid the disruption process. Chemical additives include hypotonic buffers, which cause cells to swell and burst more readily under physical shearing, lysozyme, that can be used to digest the polysaccharide component of yeast and bacterial cell walls, detergents, used to break the lipid barrier surrounding cells by solubilizing proteins and disrupting lipid:lipid, protein:protein and protein:lipid interactions. This include also DNase that can be added to samples (and /or RNase) to reduce viscosity problems during cell lysis. Protease inhibitors can also be added for prevention of protein degradation. Alternatively, processing can be expedited by treating cells with glass beads in order to facilitate the crushing of cell walls.

The methods of present invention may comprise a step of separating the nanoparticles bound to said interacting partner of the selected material by the aid of magnetic and/or electric forces, thereby isolating said interacting partner. Accordingly, the nanoparticles taken up previously by the cells, bound to intracellular components (interacting partners) are now separated from cell material, such as permeable cells or destroyed cells. Of course, the nanoparticles are separated together with the interaction partner from cells and cell material that is unwanted for further analysis. The seperation may conveniently be performed by exploiting the properties of the nanoparticles in magnetic and/or electric fields. Accordingly, the destroyed cell material comprising the nanoparticles is guided through a magnetic field so as to immobilise the particles in this field. For example, the destroyed cell material nanoparticles comprising the interaction partner may be guided through a tube laid across a magnetic field sufficiently strong so as to immobilise the particle on the inner surface of the tube. A device for performing this step, a fixed bed reactor, is shown and described in detail in Steitz et al., 2007, Bioconjugate Chemistry, 18(5) pp 1684-1690. The entirety of this document is incorporated herein by reference.

Following immobilisation of the nanoparticles in a magnetic field, washing buffers may be guided over the immobilised particles so as to remove undesired cell material from the particles. By diminishing or removing the magnetic field, the nanoparticles, separated from cell components that are not bound to the particles, may be released so as to obtain a washed fractions of nanoparticles, comprising the nanoparticles and their interaction partner.

The interaction partners may then be separated from the nanoparticles to which they are bound by exposing the sample fractions obtained from the magnetic separation step to according conditions favourable to the release of the interaction partner from the nanoparticles. For example, if the interacting partner is a protein, the protein may be precipitated in acidic conditions, followed by washing and re-dissolving of the proteins. The proteins may then be pre-analysed and selected according to their molecular weight by SDS-PAGE gel electrophoresis.

As well, other methods as chromatography, electrophoresis or ultracentrifugation can be used for pre-purification of the nanoparticles and proteins before mass spectrometrical analysis. According to the method nanoparticles can be also present during the pre-purification step.

The present invention also relates to a method of obtaining information on characteristics of a selected material within living cells. “Information” refer to any information related to the behaviour of a selected material on the surface of the nanoparticles within intact cells. For example, information relates to the nature, identity and/or quantity of interacting partners of the nanoparticles in cells. It can also relate to the way the nanoparticle is processed within the cells, for example, how and where it is transported, and the conditions of degradation of selected material on the surface of the nanoparticle or of the nanoparticle itself

According to a preferred embodiment the obtained information on characteristics of a selected material is information on the transport of the selected component within the cells, transport mechanism underlying the transport of the selected component within the cells; and/or cellular components involved in the transport of the selected material.

The obtained information may also be a chemical library. In particular, the selected material on the particles of the present invention may be such that a plurality of cellular interacting partners bind to it, allowing for a plurality of binding partners being isolated, the plurality of binding partners constituting a library. Since the nanoparticles of the present invention may comprise two or more selected material on their surface, the number of isolated interacting partners may be increased so as to constitute the library of interacting partners. For example, the chemical library may be a protein library. The present invention may thus be advantageously be employed in the field of proteomics, such as the medium to large scale study of proteins.

In the context of the present invention, the selected material on the surface of the nanoparticles may be different materials, such as different elements interacting with cellular components.

The nanoparticles may be used for assessing, for example, the presence or absence of a component within cells. For example, the nanoparticles may be used for assessing if a particular gene is expressed by checking if the respective gene product is present in the cells. If so, nanoparticles comprising a selected material specifically binding to said gene product may be used.

As a consequence, the present invention may be used to diagnose the absence and/or presence of a condition, such as a disease. The diagnosis may be made in view of the cellular components isolated with specifically designed nanoparticles, which cellular components stand for the presence or absence of a condition. A “condition”, for the purpose of the present invention, includes the status of a particular disease, location, stress, a moment in the cell cycle, a moment in development, the state of apoptosis/necrosis for example. For example, a condition of stress may be diagnosed if the presence of stress-induced proteins, for example heat shock proteins, is assessed by the aid nanoparticles binding to such proteins. For example, a moment in cell development can be assessed by the distribution of nanoparticles within the cells as determined by imaging.

The present invention also provides a method of delivering a selected material to within living cells. For example, when the selected material is a drug, for example a peptide drug, and/or a nucleotide sequence, such as DNA or RNA, for example, the drug may be delivered to the cells by exposing cells to the nanoparticles comprising the drug/nucleotide sequence. The present invention also applies to the treatment of humans and/or animals. In the latter case, a suspension comprising the nanoparticles of the invention and, as a selected material, a substance to be delivered, may be parenterally administered by injection or infusion of the suspension. Such parenteral administration may be intravenous, intramuscular, subcutaneous, for example. Of course, other ways of administration, such as topic or enteral may also be employed. Thanks to the magnetic, paramagnet and/or superparamagnetic properties of the nanoparticles of the present invention, the nanoparticles may be targeted to specific cells, such as to cells of an organ or tissue, by exposing living cells to a magnetic field. For example, when the nanoparticles are administered to a human or animal subject, an organ and/or a body part may be exposed to a magnetic filed sufficiently strong so as to immobilise or accumulate the nanoparticles in that particular organ or body part. Accordingly, the nanoparticles may be targeted to specific limps, such as to a forearm, a thigh and/or another body part of an individual.

The nanoparticles of the present invention preferably have a mean diameter of <500 nm (nanometer), more preferably <350 nm, <300 nm, <250 nm, and most preferably <200 nm, <150 nm and even <100 nm “Mean diameter” refers to the arithmetic mean. Particles with narrow size distribution are preferred. A narrow size distribution is found where the SPAN fulfils the following condition: SPAN=(D_{0.9}−D_{0.1})/D_{0.5}<1. The “span” measures the width of the particle size distribution as described in the British Standards (British Standards #BS 2955, British Standards Institution, London, UK, 1993.). Span=[particle diameter at 90% cumulative size]−[particle diameter at 10% cumulative size]/[particle diameter at 50% cumulative size]. Hence, a small span value indicates a narrow particle size distribution. Sizes are determined at 90° on a photon correlation spectrometer (PCS) from Brookhaven equipped with a BI-9000AT digital autocorrelator as set out in Example 5. Sizes are as well determined by transmission and scanning electron microscopy. As well, other techniques as magnetic measurements, surface area measurements or X-ray diffraction yield particle sizes and distributions.

The nanoparticles comprise a magnetic, paramagnetic or superparamagnetic core. The core has the property of being susceptible to a magnetic field. More particular, the core has the property of being attracted or pushed away by a magnetic field. Preferably, the core provides the nanoparticle's capacity of being immobilizable in a magnetic field. Any material providing these properties may be useful in the preparation of the nanoparticles of the present invention. For example, the core may comprise iron oxide, and/or mixed oxides containing Fe and at least one metal selected from Co, Ni, Cu, Mn, Eu, Y, and/or metal having suitable magnetic properties in general, or metal alloys, for example alloys containing Fe, Pt, Ni, Co, and/or Cu.

The preparation of cores for the nanoparticles of the present invention may be performed as disclosed previously. For example, Steitz et al (2007) disclose the preparation of iron oxide particles by alkaline coprecipitation of ferric and ferrous chlorides in aqueous solution. WO 2006/125452 and some of the publications cited above with respect to the problem to be solved also disclose commercially obtainable or synthesized nanoparticles comprising magnetic properties suitable for the purpose of the present invention.

In a further step, at least one selected material is attached to the core. More specifically, the selected material may be attached directly to the core, or to an intermediate coating providing a layer on the core. The intermediate coating, if present, preferably comprises a stabilizer material having the purpose of stabilizing the nanoparticles in suspension. In particular, the stabilizer material providing an intermediate coating in the nanoparticles of the present invention has the function of increasing steric repulsion and/or adjusting the zeta potential of the nanoparticles, thus avoiding flocculation or coagulation when suspended. The intermediate stabiliser layer is preferably used if the particle without such stabiliser could not be used to prepare a stable suspension. Accordingly, in a preferred embodiment, the particles further comprise an intermediate coating on said core, and wherein the selected material is attached to said intermediate coating at the outer surface of the particle.

On the other hand, in case that the selected material, or one of several selected materials, already provides sufficient steric repulsion to the particle, a specific stabiliser may no longer be required.

In general, however, a stabiliser layer will be required. Any suitable material may be used as a stabiliser. Also, inorganic materials, such as silanes, may be used as stabilisers. Alternatively, organic materials, for example as synthetic or natural polymers, such as polysaccharides and proteins, but also sugars may be used as stabilizers. The present inventors have made good experience with silanes, in particular aminopropyltriethoxysilane (APS). Other preferred materials are any carboxylated silanes, carboxylated molecules, including polymers, fatty acids and sugars, peptides and proteins, polysaccharides as dextran and chitosan. As well, all kind of biocompatible polymers as polyethylenglycol and polyviniylalcohol and their derivates. Biocompatible biocompatible polymers include also polystyrenes, for example poly(styrene-co-chloromethylsytrene), poly(styrene-co-chloromethylstyrene-co-methyl-4-vinylbenzyl)5 ether, poly (styrene-co-chloromethylsytrene), their derivatives and copolymers; polyphosphoester, for example poly[1,4-bis(hydroxyethyl)terephthalate-co-ethyloxyphosphate], poly((lactide-coethyleneglycol)-co-ethyloxyphosphate), poly(1,4-bis(hydroxyethyl)terephthalate-coethyloxyphosphate), their derivatives and copolymers; polyphosphazenes, for example poly(bis(4-carboxyphenoxy)phosphazene, poly(bis(1-(ethoxycarbonyl)methylamino)phosphazene), poly(bis(1-(ethoxycarbonyl)-2-phenylethylphosphazene, their derivatives and copolymers; aliphatic polyesters, for example polylactide (PLA), polyglycolide (PGA), polycaprolactone (PCL), and their copolymers, for example poly(lactide-co-caprolactone); polyhydroxybutyric acid (PHB), polyhydroxyvaleric acid (PHV), and their copolymers, for example poly(hydroxybutyric acid-co-hydroxyvaleric acid), their derivatives and copolymers; polybutylene succinates, their derivatives and copolymers.

Further biocompatible polymers considered are those containing a hydrolysable backbone, for example poly(amide-enamines), their derivatives and copolymers; poly(anhydrides), for example poly(adipic anhydride), their derivatives and copolymers; natural polymers and polysaccharides, such as cellulose, chitosan, maltodextrin, starch, agar, alginic acids and their copolymers and derivatives; polypeptides, for example gelatin, their derivatives and copolymers, poly(ethylene glycol) (PEG) based polymers, their derivatives and copolymers; polypropylene, its derivatives and copolymers, for example poly(ethylene glycol)-block-poly(propylene glycol)-block-poly(ethylene glycol); polyanhydrides, for example (4-carboxyphenoxy)propane, their derivatives and copolymers; polymers with carbon backbones, for example poly(vinyl alcohol) and poly(vinylacetate), their derivatives and copolymers; dendrimers, for example, PAMAM dendrimers, cyclotriphosphazene-PMMH-6 dendrimer, triphophosphoryl chloride, PAMAM-OS-trimethoxysilyl dendrimer, their copolymers and derivatives; hydrogels and crosslinked polymers, for example poly(acrylic acid-co-acrylamide), poly(isobutylene-comaleic acid), poly(isobutylene-co-maleic acid), poly(N-isopropylacrylamide), poly(isobutylene-co-maleic acid), poly(acrylic acid), and their derivatives; hydrophilic polymers, for example lignosulfonic acid, polyacrylamide, polyacrylic acid, poly(acrylonitrile), poly(allylamine), poly[(isobutylene), poly(4-vinylpyridine), polyvinylpyrrolidone, their derivatives and copolymers; hydrophobic polymers, for example cucurbit[x]uril, polyacrylonitrile poly(1-decene-sulfone), poly(2-ethylacrylic acid), poly(1-hexadecene5sulfone), poly(ethylene terephthalate), poly(hexafluoropropylene oxide), poly(1-hexenesulfone), poly(methyl vinyl ether), poly(1-octene-sulfone), poly(propylene glycol), poly(propylene glycol)diglycidyl ether, poly(propylene glycol)methacrylate, poly(propylene glycol)monobutyl ether, poly(propylene glycol) 4-nonylphenyl ether, poly(1-tetradecene-sulfone), poly(tetrahydrofuran), poly(vinylbenzyl chloride), polyvinyl 5 chloride, poly(4-vinylphenol), poly(4-vinylpyridine-co-styrene), their derivatives and copolymers; polyaminoacids, for example polylysine, their derivatives and copolymers; polycarbonates, polysulfones, polymethacrylate, and salts of these mentioned polymers. Of course, compatible combinations of the above disclosed substances may be used. This list the general concept of the present invention.

The nanoparticles of the invention comprise, at their surface, a selected material. The use of the singular in the expression “a selected material” should not be interpreted to mean that only a single entity, such as a single molecule is provided at the surface of the nanoparticle. On the contrary, in general, there will be a plurality of entities, such as molecules, quantum dots, and so forth, be provided as selected material. Furthermore, it is an advantage of the present invention that a plurality of chemically different materials may be provided at the surface of the nanoparticle, thus providing a plurality of functionalities to the particle. For example, the selected material may be a combination of different peptides a plurality of each being attached at the surface of the particle.

The selected material may be attached directly to the core of the nanoparticle, or, if a stabiliser layer is coated on the core, it is attached to the stabiliser. For example, the selected material is attached by covalent bond to the stabiliser. If the selected material is an organic compound and is directly attached to the core, it may, for example, be attached by be attached by covalent, ionic, dipole (hydrogen bonds) interactions. For example, Fan et al., Journal of Colloid and Interface Surface Science, 266 (2003) 215-218, disclose iron oxide particles to which biotin was introduced on the surface.

If the selected material comprises a fluorescent molecule and/or quantum dots, said fluorescent molecules or quantum dot may be attached to the surface of the nanoparticle core by electrostatic interactions such as by the layer-by-layer technique, by co-encapsulation in a matrix by emulsion techniques, by covalently coupling by a cross-linker, or by vapour and plasma techniques, for example.

If the selected material is of such nature that it cannot be attached to the core, or the stabiliser layer, if present, it may be necessary to provide one or more linker units, such as a linker molecule. The linker molecule may comprise at least two functional groups, one of which allows for attachment of the linker to the nanoparticle core or to the stabilizer layer, and an other functional group allowing attachment of the selected material. If no suitable linker is available or if necessary for reasons of chemical synthesis, for example, it may be necessary to use two or more linker molecules in succession, so as to end up with free functional groups that allow for attachment of the selected material. In the examples of the present invention, a maleimide-PEG-carboxyheptyl-N-hydroxysuccinimide (M-PEG-NHS) linker was used as a linker, as it could be attached to free —NH₂ groups of the APS-coating and provided maleimide group that can covalently bind to free —SH groups of peptides and/or dyes. Of course, other linkers providing suitable groups for attaching a respective selected material to a respective intermediate layer may be selected by the skilled person in dependence of the chemical nature of the selected material and the intermediate layer.

According to an embodiment of the present invention, the selected material is selected from (i) one or more inorganic components, (ii) one or more organic components, (iii) any combination of (i) and (ii). For example, the selected material is selected from carbohydrates including sugars, peptides, including signal peptides, DNA, RNA, proteins, including antibodies, drugs, markers including fluorescent dyes, inorganic materials including quantum dots, and combinations of two or more of these.

Example for an inorganic “selected material” are zinc oxides, silica, siloxanes, titanium oxides, aluminum oxides, calcium phosphate. These materials may be interesting for toxicological studies, for example.

Preferably, the selected material provides one or more functions to the nanoparticle, which are relevant for the specific interacting partner to be isolated or information that is to be obtained. According to a preferred embodiment, the selected material is selected from ligands binding with a putative cellular target, markers, bioactive compounds, transport-mediating compounds, cellular uptake compounds, combinations of two or more of these. It is also possible that a specific organic compound functioning as a selected material provides two or more functions.

For example, the selected material may comprise a ligand. A ligand includes compounds, such as small organic compounds, peptides, proteins, and so forth, that have a specific binding partner with the cell, in particular a receptor.

The selected material may comprise a marker. The marker has the purpose of enabling the recognition of the nanoparticles, for example while in the cells, or after being removed from the cells. The marker may thus allow for tracking the nanoparticles in the cells and visualizing their position at any given time following exposure of the cells with the nanoparticles. Markers may be selected from dyes, such as fluorescent dyes, compounds comprising isotopic labels, such as radioisotopic labels, and combinations of those. Nanoparticles may comprise one or several different markers.

The selected material may comprise a bioactive compound. For the purpose of the present invention, a bioactive compound includes drugs, and compounds that entails a specific cellular reaction, having an impact of the chemical reactions taking place in the cells. For example, a bioactive compound is a compound that has an influence on gene transcription in the cell. Of course, bioactive compounds include compounds that have an influence on the survival of the cells.

According to an embodiment, the nanoparticles comprise a cell-uptake molecule at their surface. Cell up-take compounds are compounds that mediate the uptake of the nanoparticles by up-take mechanisms which are different from endocytosis. When particles are taken up by endocytosis, they are surrounded by a cell membrane which forms a cellular vesicle, which may be a phagosome, or which may comprise coat proteins. Once enclosed in the vesicle, the intracellular fate of the nanoparticles is no longer dependent on signal peptides, discussed further below. The present invention resides in the surprising up-take of nanoparticles in a way other than endocytosis, resulting in “free”, nanoparticles being introduced into the cytoplasma of cells, “free” meaning that the nanoparticles are not enclosed in vesicles by a membrane. The literature discloses a variety of compounds that bind to cell-surface components, such as channels and other transmembrane proteins, which mediate the up-take of the compound into the interior of the cell. An example of such a compound is the cyclic RGD peptide. RGD peptides are peptides that comprise the motif of Arg-Glycine-Aspartate. An example of a cRDG peptide is Arg-Gly-Asp-D-Phe-Lys(Ac-SCH2CO). Other cell up-take proteins or peptides are known, too, for example antibodies, cationic lipids, cationic polymers, liposomes, and combinations of two or more of these. Surface charge and particle size of the nanoparticles can also be adjusted to influence cell uptake.

According to an embodiment, the nanoparticles comprise, as a selected material, a transport mediating compound. Transport-mediating compounds, for the purpose of the present invention, are compounds that direct and/or mediate the transport of an entity comprising the compound within the cells, for example, the transport to and/or into specific cellular compartments, such as plastids, mitochondrions, nucleus, lysosoms, and so on. Such compounds include in particular signal peptides targeting a cellular organelle and/or compartment. Accordingly, specific peptide signal sequences are used to direct proteins from the cytosol into the endoplasmatic reticulum (ER), mitochondria, chloroplasts, and peroxisomes, and they are also used to transport proteins from the nucleus to the cytosol and from the Golgi apparatus to the ER.

It is a surprising finding of the present invention that nanoparticles according to the present invention, when equipped with corresponding signal peptides at their surface, are transported within the cell to the target indicated by the signal peptide. A large quantity of signal peptides for specific cell compartments have been identified already. For example, a short check in publicly available databases reveals over 50 signal peptide sequences alone for targeting proteins to the mitochondrion.

In the context of the present invention, the skilled person would chose the selected material, for example a specific cell-uptake compound and/or a specific signal target peptide as a function of the specific questions to be answered and or object to be studied. The present invention thus expressly encompasses nanoparticles as defined according to the invention equipped with any cell uptake compound, such as cRGD-peptide or other peptides fulfilling the same or similar functions, and/or any peptide target sequence for targeting the nanoparticles to a specific cell compartment as desired by the skilled person. This general statement also holds true for any selected material which is provided at the surface of the nanoparticles, such as markers, drugs, quantum dots and other substances of interest.

The following examples are illustrative for the principle of the present invention and by no means are intended to limit the scope of the invention.

### EXAMPLES

### Materials

Cyclic RGD peptide [Arg-Gly-Asp-D-Phe-Lys(Ac-SCH2CO) abbreviated to cRGD] was acquired from Peptides International (Louisville, Ky.). 7-Hydroxycoumarin-3-carboxylic acid N-succinimidyl ester (Coumarin-NHS) and fluorescein isothiocynate (FITC) were purchased by Sigma (St. Louis, Mo., USA). Amine reactive dyes, Alexa 405 and Alexa 488, were obtained from Invitrogen (Carlsbad, Calif., USA). The maleimide-PEG-carboxyheptyl-NHS (abbreviated to M-PEG-NHS), MW 5000 was obtained from NOF CORPORATION (Tokyo, Japan).

### Example 1

### Preparation of Cores of Superparamagnetic Iron Oxide Nanoparticles

Superparamagnetic iron oxide nanoparticles are prepared by alkaline co-precipitation of ferric and ferrous chlorides in aqueous solution as described by Chastellain M. et al., J Colloid Interface Sci 278, 353-360, 2004. The obtained black precipitate is washed several times with ultra-pure water and the remaining solid refluxed in nitric acid (10⁻² M) in the presence of iron-(III)-nitrate. The obtained brown suspension is dialyzed against 0.01 M nitric acid for two days, and stored at 4° C.

The iron oxide nanoparticle cores were characterized thoroughly by X-ray diffraction (XRD), surface area measurements (BET), transmission electron microscopy (TEM), photon correlation spectroscopy (PCS), and magnetic measurements (MM). The results are summarized in Table 1.

TABLE 1
Average particle size of bare iron oxide nanoparticles
obtained by different methods

| Method | Size [nm] |
|---|---|
| XRD average volume weighted size | 10 ± 1 |
| BET average surface weighted size | 8.9 ± 0.2 |
| TEM average number weighted size | 9 ± 2 |
| PCS average number weighted size | 13 ± 4 |

### Example 2

### Preparation of APS Coated Nanoparticles

The paramagnetic nanoparticle cores obtained in Example 1 were provided with an aminopropyltriethoxysilane (APS) coating as described by Steitz et al., 2007 Bioconjugate Chemistry DOI:10.1021/bc070100v.

In brief, superparamagnetic iron oxide nanoparticles were coated with 3-(aminopropyl)triethoxysilane (APS) via a sonochemical route. 400 μL ferrofluid (from concentrations ranging from 12 mg to 0.6 mg iron/mL) were added to 9.6 mL ethanol in a 50 mL polystyrene vessel. Subsequently 1.5 mL aqueous ammonia (volume fraction 25%) and 1 mL APS were added and the mixture was continuously sonicated for 1 h in an ultrasound horn (Ultrasons Annemasse, Sonimasse S20). The reaction was carried out at a molar ratio of 1:35:15 APS/ethanol/H₂0. After sonication the sample was centrifuged at 30,000 g for 25 minutes (Jouan, GR2022). The supernatant was discarded and the pellet was re-suspended in ultrapure water. This process was repeated three times. The magnetic particles were sedimented overnight on a permanent magnet (Maurer Magnets, Switzerland, Bᵣ=300 mT) and the clear supernatant was removed and the particles were re-dispersed in water. The particles having a mean diameter of 25±2 nm consisted of 94% of —Fe₂O₃ (w/w) and 6% aminopropyl silica (w/w) as determined by atomic emission spectroscopy and had a saturation magnetisation of 58 emu/g, which reflects the composition of the beads as the uncoated nanoparticles showed a saturation magnetisation of 61 emu/g.

### Example 3

### Synthesis of Targeting Peptides and Preparation of Thiolated Fluorophores

For the fabrication of the nuclear and mitochondrial targeting peptides with the corresponding sequences PKKKRKVGC and MALLRGVFIVAAKRTPFGAYGC, the protected amino acid derivatives were used (Novabiochem). The NovaSyn TGR resin (loading: 0.23 mmol/g), was used as the solid phase support. All solvents were dried over molecular sieves 3A (Fluka). Peptide syntheses were performed using a PSW1100 peptide synthesizer (Chemspeed AG) according to the instructions of the manufacturer. After completion, the resin was recovered and the cleavage was carried out with trifluoroacetic acid (TFA)/triisopropylsilane(TIS)/H₂O (94:5:1) for four hours at room temperature with shaking. The peptide was precipitated directly from the cleavage cocktail into cold diethyl ether (800 ml), collected using a filter, dried under vacuum, and stored at −20° C. The peptide was dissolved in 0.1% TFA in water just prior to purification. Fluorophore NHS esters were reacted with five equivalents of cysteamine. The thiolated fluorophores and peptides were purified via preparative scale reverse-phase HPLC using a Waters Ultrapurification system with a preparative Atlantis™ dC18 column (19×150 mm) and mobile phases of acetonitrile and 0.1% (v/v) TFA in water. The peptide was sequenced using ESI+ MS/MS in a Thermo Scientific LTQ Linear Ion Trap mass spectrometer equipped with an Advion Biosciences electrospray source.

### Example 4

### Functionalization of APS Coated Nanoparticles Using Magnetic Bed Reactor

### Example 4.1

### First Functionalization with Malieimide-poly(ethylene glycol)-carboxyheptyl-N-hydroxysuccinimide (M-PEG-NHS)

The nanoparticles comprising an APS coating obtained in Example 2 was dissolved in 20 mM borate buffer, pH 7.5, at final concentration of 5 mg/ml. The APS-coated nanoparticles were immobilized in a Magnetic Bed Reactor as disclosed in Steitz et al., 2007 Bioconjugate Chemistry 18(5) pp 1684-1690, illustrated schematically in FIG. 2. In order to create a high magnetic field gradient, 4 magnet assemblies, each consisting of a stack of 5 FeNdB magnets with a resulting surface remanence magnetization of 0.66 T, were placed repulsive to each other. An INOX bloc was used as the support for the magnet arrangement. The distance between the magnets was controlled by screws leaving space for the insertion of a tube in the core of the reactor. High gradients of several hundred Tm⁻¹ were generated with this magnetic arrangement. Within 10 millimeters of distance from a single stack of 5 single magnets a gradient of 55 T/m was measured.

In a first step, 2 mg of immobilized APS coated nanoparticle cores were functionalized by recirculating 4 mg of the heterobifunctionalized M-PEG-NHS over a milligram of immobilized particles on the softmagnetic iron-nickel wire in the magnetic bed reactor. The M-PEG-NHS solution was recirculated for 20 minutes at a flow rate of 3.5 ml/min. After the reaction of the N-hydroxysuccinimide site of the PEG to the amino groups of the APS-nanoparticles the derivatized particles were continuously washed with 150 ml of 20 mM borate buffer at pH 6.8. Following the washing step, immobilized M-PEG-APS-nanoparticles were further functionalized by coupling thiolated moieties of specific molecules of interest to maleimide groups of PEG.

### Example 4.2

### Specific Functionalization of Nanoparticles

Peptides and thiolated flurophores (Fluoresceinisothiocyanate, FITC) were dissolved in 20 mM borate buffer at pH 6.8. Prior functionalization, 1 mg of cRGD (Peptides International) was deprotected using 10 μl of 0.5 M sodium methoxyde and diluted in borate buffer.

### 4.2.A Nanoparticles Comprising Nuclear Targeting Peptide, cRGD Cell-Uptake Peptide and Fluorescent Dye (NTP-cRGD-nanoparticles)

In order to obtain fully functionalized nuclear targeting nanoparticles (NT-nanoparticles), 1.5 mg of PKKKRKVGC, 0.1 mg of cRGD and 0.1 mg of FITC-SH were premixed and added to the immobilized M-PEG-APS-nanoparticles in fixed bed reactor.

### 4.2.B Nanoparticles Comprising Mitochondrial Targeting Peptide, cRGD Cell-Uptake Peptide and Fluorescent Dye (MTP-cRGD-nanoparticles)

In an analogous way, mitochondrial targeting nanoparticles were obtained by treatment of 1.5 mg of MALLRGVFIVAAKRTPFGAYGC, 0.1 mg of deprotected cRGD and 0.1 mg of 7-coumarin-SH (Sigma-Aldrich). To obtain corresponding positive and negative controls for these particles either cRGD or nuclear targeting peptide or both were omitted from the synthesis.

Then each of the mixtures described above was recirculated for one hour and washed as it was aforementioned. Following the wash, active maleimide groups were quenched by adding one milliliter of 4 mg/ml cysteine (Acros Organics) to both reaction and allowed to react for two hours again with either recirculation. After quenching, the mixture was washed once more and functionalized nanoparticles were released from the nickel wire.

For the quantification of the fluorescence in the supernatant and on the nanoparticle surfaces, fluorescence standard curves with the Alexa dye were measured at an excitation wavelength of 578±9 nm and an emission wavelength of 605±20 nm

FIG. 4 shows the chemical structure of the surface of the nanoparticles functionalised with MTP-target peptide, cRGD peptide for uptake by the cell and FITC dye as selected materials.

### Example 5

### Size and Zeta Potential Determination

Light-scattering measurements were carried out at 90° on a photon correlation spectrometer (PCS) from Brookhaven equipped with a BI-9000AT digital autocorrelator. The CONTIN method was used for data processing. The concentration of iron oxide nanoparticles was set to 0.025 mg iron/ml for all measurements. The theoretical refractive index of magnetite (2.42) was used to calculate the number-weighted distribution from the raw-intensity weighted data. Zeta-potential measurements were performed using the same setting, equipped with a platinum electrode. The electrode was cleaned for ten minutes in an ultrasonic bath prior to each measurement and pre-equilibrated for two minutes in an aliquot of the sample. For size and zeta-potential the nanoparticles were diluted in a 20 mM borate buffer solution, pH 7.5. Viscosity, refractive index, and dielectric constant of pure water were used to characterize the solvent. The size distribution of the functionalized nanoparticles prepared in Example 4 is shown in FIG. 3. As can be seen, the particle size increases upon (a) MTP coupling and (b) cRGD coupling:
Mean size APS-Core: 25±2 nm
Mean size MTP-Core: 78.7±0.7 nm
Mean size MTP-cRGD-Core: 105±2 nm

### Example 6

### Cell Culture and Treatments

HELA (human cervix carcinoma cells) cells were grown in DMEM medium (Gibco-BRL, Invitrogen, Denmark) supplemented with 10% fetal calf serum (Gibco-BRL, Invitrogen, Denmark) and 1% penicillin/streptomycin (Gibco-BRL, Invitrogen, Denmark). For iron quantification assay, one day prior experiment, the cells were detached in trypsin-EDTA (Gibco-BRL, Invitrogen, Denmark) and grown in complete medium in 48-well plates (Costar) at ˜10⁴ cells per well. For fluorescence imaging ˜10⁵ cells were placed in 30 mm cell culture wells (Fluorodish, WPI, London, UK). On the day of the experiment, the dilutions of nanoparticles were added at the designated concentrations and for indicated time, followed by further experimental manipulations.

### Example 7

### Live Cell Imaging an Image Analysis

200 μL of functionalized nanoparticle suspension containing 1 mg of iron were added into 30 mm cell culture wells (WPI, UK) under standard conditions and were incubated for 10 h. Just prior to microscopic observation, Hoechst 33342 (Sigma) dye was added in a final concentration of 1 μg/mL to the cells which were incubated with nuclear targeting nanoparticles. After incubation, cells were washed three times with PBS and 2 ml of OPTIMEM medium (Gibco) was added in cell culture dish. Photomicroscopic imaging was conducted on a Zeiss LSM 510 META confocal microscope using a 63× oil immersion objective and a z-direction step of 1 μm. Confocal image projections were processed with the Zeiss LSM image browser and Adobe Photoshop. IMARIS software (BITPLANE, MN, USA) was used to determine number of the nuclear targeting nanoparticles present in the nucleus. Briefly, the presence of the Hoechst 33342 dye in nucleus was used for surface rendering, whereas labeled nuclear targeting nanoparticles were assigned to the particles with fluorescent diameter of 0.3 μm. The particles placed inside rendered nuclear surface were marked as the nuclear, whereas those placed outside the surface were denoted as cytoplasmic. To calculate colocalization correlation between mitochondrial targeting nanoparticles and mitochondria, METAMORPH software was used.

### Example 8

### Determination of Iron Uptake by Cells

For iron quantification assay, one day prior experiment, HELA cells were detached in trypsin-EDTA (Gibco-BRL, Invitrogen, Denmark) and grown in complete medium in 48-well plates (Costar) at ˜10⁴ cells per well. The cells were incubated at 90% confluency with different nanoparticle types, in particular APS-coated nanoparticle cores (APS-nanoparticles), cRGD-nanoparticles, MTP-nanoparticles, MTP-cRGD-nanoparticles, NTP-nanoparticles, and NTP-cRGD-nanoparticles (all of them containing fluorescent dye) at concentrations of 100 and 20 μg/ml in 200 μl of DMEM medium (Gibco-BRL, Invitrogen, Denmark) supplemented with 10% fetal calf serum for 2, 6, and 24 h. In order to remove the non-internalized nanoparticles, cells were washed after incubation with 250 μl PBS, 250 μl DMEM supplemented with 10% fetal calf serum and again with 250 μl PBS. Each washing step followed a subsequent incubation step for 1 min on a vortex. After the washing procedure the cell layer was dissolved for 1 h in 6N HCl (125 μl/well of a 48-well plate), then 125 μl of a 5% solution of K₄[Fe(CN)₆] (Merck) in H₂O was added and the absorbance was read after 10 min at 690 nm in a multiwell plate reader (Infmite 200, Tecan). A standard curve using the differently coated iron oxide nanoparticles at concentrations ranging from 0.25 to 50 μg/ml was recorded in the same conditions to quantify the amount of cell-bound iron. Each experiment was repeated in triplicate wells at least three times. Means and standard deviation were calculated. It is important to note that the different particles were incubated in the corresponding media for one hour prior to cell exposure.

Incubation of APS-nanoparticles resulted in saturation already after 2 h incubation time. Considerably higher particle concentrations showed no saturated uptake even after 24 h incubation. NTP-nanoparticles and NTP-cRGD-nanoparticles showed generally lower uptake compared to the APS-nanoparticles. Uptake was increased by longer incubation times.

### Example 9

### Separation of Magnetically Bound Protein

Hela cells were grown in a Petri dish containing 10⁷ cells. Prior incubation with nanoparticles, cells were washed with 10 ml PBS. Than DMEM supplemented with 10% fetal calf serum containing 100 μg/ml of MTP-cRGD-nanoparticles or NTP-cRGD-nanoparticles, respectively, was added and incubated overnight. To remove the adsorbed nanoparticles, cells were washed after incubation with 10 ml PBS, 10 ml DMEM supplemented with 10% fetal calf serum and again with 10 ml PBS. Each washing step followed a subsequent incubation step for 1 min on a vortex. Again washing was done with 10 ml of cool PBS (4° C.) and the plate was put on ice. After removing of the PBS 700 μl of nuclear or mitochondrial lysis buffer was added and the cells were scraped of the Petri dish using a rubber scraper. Nuclear lysis buffer, pH 7.4, was containing 250 mM sucrose, 25 mM KCl, 5 mM MgCl₂, 10 mM TrisHCl and 1 tablet of protease inhibitor per 50 ml of buffer (KEEP-IT-EASY, Roche). Mitoiso 2 (Sigma-Aldrich) was used as mitochondrial lysis buffer. The detached cells were lysed by push and pulling the cells for approximately 10 times through a 0.45×12 mm needle (Braun, Sterican) by using a 1 ml syringe. The quality of the disintegrated cells was assessed by trypan blue staining under the microscope. Thereafter, the cellular extract was put at 4° C. into the magnetic reactor, as described for the synthesis of the particles (Steitz et al.), and incubated for 10 minutes with stopped flow. The magnetic sediment on the column was washed with 4 ml of nuclear lysis buffer and than a series of subsequent precooled (4° C.) KCl concentrations (50 mM, 200 mM, 1 M, 1 M+0.05% SDS) each step with a volume of 2 ml was passed over the column. The eluted fractions were collected and stored at −80° C.

Control experiments were performed with cell lysate without previous incubation of the Hela cells with nanoparticles. Lysis of the cells was performed as described before. 500 μl of the cell lysate was incubated with 50 μg/ml of MTP-cRGD-nanoparticles or NTP-cRGD-nanoparticles, respectively. Then magnetic separation was done according to the previous procedure.

### Example 10

### Protein Precipitation

The sample fractions taken from the magnetic separation were defrosted ice (4° C.). To 1 ml of each sample 250-300 μl of 100% trichloroacetic acid (Sigma-Aldrich) were added. Afterwards, the precipitate was centrifuged for 30 min at 18.000 g at 4° C. and the supernatant was discarded. The pellet was washed 3 times in ice cold acetone followed by a subsequent centrifugation step and discarding of the supernatant. The protein samples were re-dissolved by addition of 50 μl of rehydration buffer (7 M urea, 2 M thiourea, 20 mg/ml 3-[(3-cholamidopropyl)dimethylammonio]-1-propanesulfonate, 15 mg/ml DL-dithiothreitol, 10 μl/ml IPG buffer) and analyzed by SDS-PAGE gel electrophoresis. FIG. 5 shows an illustrative SDS-PAGE gel following separation of proteins by electrophoresis.

### Example 11

### Protein Identification

The collected protein fractions were separated by SDS-PAGE with a 10% gel and stained with Coomassie. For mass spectrometry, the bands were excised and samples were treated by mixture of dithioerythritol and alkylated iodoacetamide in order to reduce and block disulfide bonds. Samples were then dried and in-gel digested by sequencing-grade trypsin (Promega AG) for at least 12 hours at 37° C. The supernatant was collected and fresh NH₄HCO₃ solution was added. The gel plugs were incubated in the buffer for 20 min at room temperature. The supernatant was collected and a solution containing 50% ethanol and 5% formic acid was added to cover the gel pieces. The gel pieces were incubated in the buffer for 20 min at room temperature and the supernatant collected. The last elution procedure was repeated twice prior to pooling all supernatants. The combined supernatants were concentrated using a SpeedVac to approximately complete dryness and resuspended in acetonitrile. Samples were separated by reverse phase liquid chromatography at a flow rate of 400 nl/min on an Agilent Chip C18 capillary column (75 μm id×43 mm) MS analysis was performed on a Bruker HCT Ion-Trap instrument. The MSDB human sub-database was used to map the fingerprints with specific identification predicted through the Mascot (Matrix Science) or Phenyx search engine (GeneBio). Table 2 further below shows the protein library obtained from the proteins isolated with the MTP-cRGD-nanoparticles after magnetic extraction. FIG. 6 provides an illustration of the cellular relations of the proteins of the database.

### Examples 12 and 13

### PVA and Vinyl Alcohol/Vinyl Amine Copolymer Coated Nanoparticles

The magnetic nanoparticle cores obtained in Example 1 were stabilized by either pure polyvinyl alcohol (PVA) and by vinyl alcohol/vinyl amine copolymer instead of APS (Example 2). Accordingly, polymer solutions were prepared by dissolving the powders in water, rapidly heating the solutions for 15 min at 90° C., and filtering the hot solutions over paper filters (Schleicher & Schuell AG). Ultra-pure deionized water (Seralpur delta UV/UF setting, 0.055 μS/cm) was used in all synthesis steps. The magnetic nanoparticle cores obtained in Example 1 were mixed at various ratios with the polymer solutions. Polymer chains were cross-linked using 0.1% glutaraldehyde. Cross-linking was accomplished overnight under shaking in a polystyrene tube.

### Example 14

### Immobilization of Functional Proteins to Vinyl Alcohol/Vinyl Amine Copolymer Coated Nanoparticles

A suspension of nanoparticles (1 mg Fe/ml) stabilized by a vinyl alcohol/vinyl-amine copolymer coating containing 0.1% (v/v) glutaraldehyde as cross linking reagent (Example 13) were functionalized with selected materials. The suspension was loaded in the magnetic fixed bed reactor using a Ni/Fe wire as support. The not immobilized polymer coated nanoparticles were removed from the reactor with 6 ml PBS at a flow rate of 2 ml/min. The amine groups of the coated nanoparticles were used for further coupling with an antibody. Monoclonal Rat anti-mouse CD11b was dissolved in 0.5M sodium carbonate, pH 9.5, to a final concentration of 50 μg/ml and reacted for 1 hour at a flow rate of 2 ml/min in the reactor. The unreacted components were removed by washing with 10 ml PBS at the same flow rate. Finally, 0.2M lysine in 0.5M sodium carbonate solution, pH 9.5, was allowed to react for 1 hour in order to block excess reactive sites. Functionalized particles were then removed from the reactor and suspended in PBS for further biological application.

TABLE 2
Proteins Identified in Combination with MTP-cRGD-Nanoparticles after Magnetic Extraction

| | | | Relation with | Accession | Theoretical | Peptides | Sequence |
|---|---|---|---|---|---|---|---|
| Localization | Gene name | Protein | mitochondria* | number | Mr (KDa) | sequenced | coverage (%) |
| Mitochondria | HSP75 | Heat shock protein 75 kDa | | Q12931 | 80 | 2 | 2 |
| | HSPD1 | 60 kDa heat shock protein | | P10809 | 58 | 6 | 17 |
| | ATP5A1 | ATP synthase subunit alpha, | | P25705 | 56 | 5 | 10 |
| | | (EC 3.6.3.14) | | | | | |
| | ATP5B | ATP synthase subunit beta, | | P06576 | 51 | 9 | 29 |
| | | mitochondrial precursor | | | | | |
| | | (EC 3.6.3.14). | | | | | |
| | MDH2 | Malate dehydrogenase | | P40926 | 33 | 2 | 6 |
| | | (EC 1.1.1.37). | | | | | |
| Cytosolic | HSP90AA1 | Heat shock protein HSP 90-alpha | Interacts with | P07900 | 84 | 8 | 13 |
| chaperones | | | mitochondrial | | | | |
| | | | HSP60 | | | | |
| | HSP90AB1 | Heat shock protein HSP 90-beta | Interacts with | P08238 | 83 | 3 | 19 |
| | | | TOM34¹⁾ | | | | |
| | HSPA1A | Heat shock 70 kDa protein 1 | Interacts with | P08107 | 70 | 6 | 11 |
| | HSPA2 | Heat shock-related 70 kDa | mitochondrial | P54652 | 70 | 4 | 5 |
| | | protein 2 | HSP60; in | | | | |
| | HSPA6 | Heat shock 70 kDa protein 6 | complex with | P17066 | 70 | 3 | 5 |
| | HSPA5 | Heat shock 70 kDa protein 5 | HSP90-alpha | P11021 | 70 | 2 | 3 |
| | HSPA7 | Heat shock 70 kDa protein 7 | | P48741 | 70 | 1 | 5 |
| | | [Fragment] | | | | | |
| | HSPA8 | Heat shock 70 kDa protein 8 | | P11142 | 75 | 10 | 16 |
| | HSPA1L | Heat shock 70 kDa protein 1L | | P34931 | 70 | 5 | 8 |
| Cytosolic | CCT6A | T-complex protein 1 subunit zeta | 2) | P40227 | 58 | 1 | 2 |
| chaperonins | CCT2 | T-complex protein 1 subunit beta | | P78371 | 57 | 1 | 1 |
| Cytoskeleton | TUBB | Tubulin beta chain | 3) | P07437 | 50 | 7 | 19 |
| | TUBB2C | Tubulin beta-2C chain | | P68371 | 50 | 5 | 15 |
| | TUBB3 | Tubulin beta-3 chain | | Q13509 | 50 | 5 | 15 |
| | TUBA1B | Tubulin alpha-ubiquitous chain | | P68363 | 50 | 6 | 16 |
| | TUBB2A | Tubulin beta-2B chain | | Q9BVA1 | 50 | 4 | 13 |
| | TUBB2A | Tubulin beta-2A chain | | Q13885 | 50 | 4 | 13 |
| | TUBB4 | Tubulin beta-4 chain | | P04350 | 50 | 4 | 12 |
| | TUBA1A | Tubulin alpha-3 chain | | Q71U36 | 50 | 5 | 13 |
| | TUBA1C | Tubulin alpha-6 chain | | Q9BQE3 | 50 | 5 | 13 |
| | TUBB6 | Tubulin beta-6 chain | | Q9BUF5 | 50 | 3 | 9 |
| | TUBA3D | Tubulin alpha-2 chain | | Q13748 | 50 | 4 | 9 |
| | TUBA4A | Tubulin alpha-1 chain | | P68366 | 50 | 2 | 6 |
| | TUBB4Q | Tubulin beta-4q chain. | | Q99867 | 50 | 2 | 6 |
| | FSCN1 | Fascin | | Q16658 | 55 | 3 | 8 |
| | ACTB | Actin, cytoplasmic 1 | | P60709 | 41 | 3 | 13 |
| | ACTG1 | Actin, cytoplasmic 2 | | P63261 | 41 | 3 | 13 |
| | VIM | Vimentin | | P08670 | 53 | 3 | 9 |
| | ACTN4 | Alpha-actinin-4 | | O43707 | 104 | 5 | 6 |
| Sugar | TKT | Transketolase | Expected to be | P29401 | 68 | 6 | 11 |
| metabolism | PKM2 | Pyruvate kinase isozymes | at the cytosolic | P14618 | 57 | 4 | 11 |
| | | M1/M2 | side of | | | | |
| | ENO1 | Alpha-enolase | mitochondrial | P06733 | 47 | 9 | 23 |
| | ENO3 | Beta-enolase | membrane | P13929 | 49 | 3 | 8 |
| | ENO2 | Gamma-enolase | | P09104 | 47 | 2 | 4 |
| | PGK1 | Phosphoglycerate kinase 1 | | P00558 | 44 | 2 | 5 |
| | AKR1C1 | Aldo-keto reductase family 1 | | Q04828 | 37 | 1 | 3 |
| | | member C1 | | | | | |
| | AKR1C2 | Aldo-keto reductase family 1 | | P52895, | 37 | 1 | 3 |
| | | member C2 | | | | | |
| | AKR1C3 | Aldo-keto reductase family 1 | | P42330 | 37 | 1 | 3 |
| | | member C3 | | | | | |
| | AKR1C4 | Aldo-keto reductase family 1 | | P17516 | 37 | 1 | 3 |
| | | member C4 | | | | | |
| | LDHA | L-lactate dehydrogenase A chain | | P00338 | 36 | 1 | 3 |
| | TALDO1 | Transaldolase | | P37837 | 38 | 1 | 3 |
| | GAPDH | Glyceraldehyde-3-phosphate | | P04406 | 36 | 8 | 22 |
| | | dehydrogenase | | | | | |
| Translation | EEF1A1 | Elongation factor 1-alpha 1 | | P68104 | 50 | 2 | 8 |
| machinery | EEF1D | Elongation factor 1-delta | | P29692 | 30 | 2 | 9 |
| | EEF1G | Elongation factor 1-gamma | | P26641 | 50 | 2 | 6 |
| | NACA | Nascent polypeptide-associated | | Q13765 | 23 | 1 | 7 |
| | | complex subunit alpha ( | | | | | |
| | RPLP0 | 60S acidic ribosomal protein P0 | | P05388 | 34 | 3 | 17 |
| | RPSA | 40S ribosomal protein SA | | P08865 | 32 | 2 | 10 |
| | TARS | Threonyl-tRNA synthetae | | P26639 | 83 | 2 | 3 |
| Endoplasmatic | HSP90B1 | Heat shock protein 90 kDa beta | | P14625 | 90 | 6 | 8 |
| reticulum | | member | | | | | |
| | SERPINH1 | Serpin H1 precursor | | P50454 | 46 | 2 | 7 |
| plasma | ANXA1 | Annexin A1 | | P04083 | 38 | 4 | 11 |
| membrane | ANXA2 | Annexin A2 | | P07355 | 38 | 7 | 20 |
| Nucleus | NCL | Nucleolin | | P19338 | 76 | 2 | 4 |
| | SEPT2 | Septin-2 | | Q15019 | 41 | 1 | 4 |
| | HNRPA2B1 | Heterogeneous nuclear | | P22626 | 36 | 1 | 3 |
| | | ribonucleoproteins A2/B1 | | | | | |

¹⁾mitochondrial receptor subunit;
2) Interacts with mitochondrial HSP60;
3) Interacts with mitochondrial HSP60;

1.-25. (canceled)

26. A method of isolating one or more interacting partners of one or more selected material, said method comprising the steps of:
exposing living cells to nanoparticles, wherein said nanoparticles:
have a mean diameter of <500 nm,
comprise a magnetic, paramagnetic or superparamagnetic core, and,
comprise, at a surface of said nanoparticle, said selected material;
removing said nanoparticles from said cells following exposure to the nanoparticles; and
separating the nanoparticles bound to said interacting partner of the selected material by the aid of magnetic and/or electric forces, thereby isolating said interacting partner.

27. The method of claim 26, wherein the particles further comprise an intermediate coating on said core, and wherein the selected material is attached to said intermediate coating at the outer surface of the particle.

28. The method of claim 27, wherein the intermediate coating comprises a stabilizer material.

29. The method of claim 26, wherein the step of exposing cells to said nanoparticles comprises the step of adding a suspension of nanoparticles to said cells.

30. The method of claim 26, wherein the selected material is selected from carbohydrates including sugars, peptides including signal peptides, DNA, RNA, proteins including antibodies, drugs, markers including fluorescent dyes, inorganic materials including quantum dots, and combinations of two or more of these.

31. The method of claim 26, wherein said nanoparticles comprise, at their surface, a signal peptide targeting a cellular organelle and/or compartment.

32. The method of claim 26, wherein said nanoparticles comprise a cell-uptake molecule.

33. The method of claim 26, wherein the nanoparticles comprise, at their surface, a bioactive compound.

34. The method of claim 26, wherein the selected material is selected from:
ligands binding with a putative cellular target;
marker molecules;
bioactive compounds;
transport-mediating compounds;
cellular uptake compounds
combinations of two or more of these.

35. The method of claim 26, wherein the step of exposing living cells to nanoparticles comprises a step selected from (i) incubating cells in vitro with said nanoparticles, and (ii) administrating said nanoparticles to a human or animal subject.

36. The method of claim 26, wherein the step of exposing living cells to nanoparticles is conducted for a time which is sufficiently long so as to allow for uptake of the nanoparticles within the cells and, preferably, for interaction of the selected material with intracellular cell components to occur.

37. The method of claim 26, wherein the step of removing said nanoparticles from said cells comprises the steps of destroying, lysing, and/or increasing the cell-membrane permeability of said cells.

38. The method of claim 26, further comprising the step of constituting a library of said interacting partners.

39. The method of claim 26, wherein the one or more interacting partners are one or more cellular components.

40. A method of diagnosis of a presence and/or an absence of a condition, the method comprising the steps of:
a) comparing the chemical library obtained by the method of claim 38 to a standard library representing a specific condition; and,
b) diagnosing from similarity between the chemical library obtained by said method of claim 38 and the standard library the presence and/or the absence of said condition.

41. The method of claim 26, further comprising the step of studying and/or characterising cell components interacting with said selected material, thereby obtaining information on characteristics of the selected material within cells.

42. The method of claim 41, wherein the obtained information on characteristics of a selected material is information on:
the transport of the selected component within the cells;
transport mechanism underlying the transport of the selected component within the cells; and/or
cellular components involved in the transport of the selected material.

43. A method of delivering a selected material into living cells, the method comprising the steps of:
exposing living cells to nanoparticles, wherein said nanoparticles:
have a mean diameter of <500 nm,
comprise a magnetic, paramagnetic or superparamagnetic core, and,
comprise, at a surface of said nanoparticle, said selected material.

44. The method of claim 43, comprising the step of exposing the living cells to a magnetic field.

45. Nanoparticles comprising a magnetic, paramagnetic and/or superparamagnetic core and, at a surface of the nanoparticles, at least a first and a second selected material, wherein the first and the second materials are different.

46. The nanoparticles of claim 45, containing three (3) or more different selected materials on the surface.
